(19) United States
(12) Patent Application Publication
(10) Pub. No.: US 2026/0165572 A1
(43) Pub. Date: Jun. 18, 2026

(54) ENDOSCOPE REPROCESSOR, CONTROL METHOD, AND CONTROL APPARATUS FOR PRESSURE-CONTROLLED FLUID SUPPLY TO AN ENDOSCOPE

(71) Applicant: OLYMPUS MEDICAL SYSTEMS CORP., Tokyo (JP)

(72) Inventor: Ryo SATO, Tokyo (JP)

(73) Assignee: OLYMPUS MEDICAL SYSTEMS CORP., Tokyo (JP)

(21) Appl. No.: 19/533,827

(22) Filed: Feb. 9, 2026

Related U.S. Application Data
(63) Continuation of application No. PCT/JP2023/029252, filed on Aug. 10, 2023.

(51) Int. Cl. A61B 1/12; A61B 1/015

(52) U.S. Cl. CPC A61B 1/125; A61B 1/015

(57) ABSTRACT

An endoscope reprocessor includes a first tube connectable to a first ferrule of an endoscope, a second tube connectable to a second ferrule of the endoscope, a fluid supply assembly configured to supply fluid to a conduit of the endoscope via the first tube and the second tube, and a controller. The conduit includes a first conduit communicating with the first ferrule, and a second conduit communicating with the second ferrule, the first and second conduits converging into a third conduit having an opening at a distal end of an insertion portion of the endoscope. The controller is configured to control the fluid supply assembly to feed liquid to the first and second conduits at a first pressure, and then feed liquid to the first and second conduits at a second pressure higher than the first pressure.

### CROSS REFERENCE TO RELATED APPLICATION

This application is a continuation of International Application No. PCT/JP2023/029252, filed on Aug. 10, 2023, the entire contents of which are incorporated herein by reference.

### FIELD

The present disclosure relates to an endoscope reprocessor, a control method, and a control apparatus for pressure-controlled fluid supply to an endoscope.

### BACKGROUND

For endoscopes used in the medical field, a reprocessing process, such as cleaning and disinfection, is essential for reuse after an insertion portion is inserted into a subject to perform intrasubject observation and treatment with a treatment instrument.

An endoscope reprocessor removes contaminants such as blood clots and mucus adhering to a conduit by connecting a connector of a tube to each of a suction ferrule and a forceps ferrule of an endoscope, supplying a fluid to each tube, and discharging the fluid from an opening at a distal end of an insertion portion.

International Publication No. WO2015-001843 discloses an endoscope cleaning apparatus that cleans a forceps ferrule using liquid that leaks from a space between the forceps ferrule and a connector of a tube connected to the forceps ferrule.

International Publication WO2016-194456 discloses an endoscope reprocessor that performs flow control to adjust a flow rate of a first fluid supply unit that supplies liquid as a fluid and a flow rate of a second fluid supply unit that supplies gas as a fluid.

### SUMMARY

According to aspects of the present disclosure, an endoscope reprocessor is provided, which includes a first tube, a second tube, a fluid supply assembly, and a controller. The first tube is connectable to a first ferrule of an endoscope. The second tube is connectable to a second ferrule of the endoscope. The fluid supply assembly is configured to supply fluid to a conduit of the endoscope via the first tube and the second tube. The conduit includes a first conduit communicating with the first ferrule, and a second conduit communicating with the second ferrule, the first and second conduits converging into a third conduit having an opening at a distal end of an insertion portion of the endoscope. The controller is configured to control the fluid supply assembly to feed liquid to the first and second conduits at a first pressure, and then feed liquid to the first and second conduits at a second pressure higher than the first pressure.

According to aspects of the present disclosure, further provided is a control method implementable by an endoscope reprocessor configured to supply fluid to a conduit of an endoscope via a first tube and a second tube. The method includes feeding liquid to a first conduit and a second conduit at a first pressure, and then feeding liquid to the first conduit and the second conduit at a second pressure higher than the first pressure. The endoscope includes a first ferrule, a second ferrule, and the conduit. The first ferrule is connectable to the first tube. The second ferrule is connectable to the second tube. The conduit includes the first conduit communicating with the first ferrule, and the second conduit communicating with the second ferrule, the first and second conduits converging into a third conduit having an opening at a distal end of an insertion portion of the endoscope.

According to aspects of the present disclosure, further provided is a control apparatus that includes a connection interface and a processor. The connection interface is connectable to a fluid supply assembly configured to supply fluid to a conduit of an endoscope via a first tube and a second tube. The processor is configured to control the fluid supply assembly to feed liquid to a first conduit and a second conduit at a first pressure, and then feed liquid to the first and second conduits at a second pressure higher than the first pressure. The endoscope includes a first ferrule, a second ferrule, and the conduit. The first ferrule is connectable to the first tube. The second ferrule is connectable to the second tube. The conduit includes the first conduit communicating with the first ferrule, and the second conduit communicating with the second ferrule, the first and second conduits converging into a third conduit having an opening at a distal end of an insertion portion of the endoscope.

### BRIEF DESCRIPTION OF DRAWINGS

FIG. 1 is a perspective view of an endoscope reprocessor according to a first embodiment.

FIG. 2 is a configuration diagram of a main part of the endoscope reprocessor according to the first embodiment.

FIG. 3 is a cross-sectional view of a joint section between a first connector and a forceps ferrule in the endoscope reprocessor according to the first embodiment.

FIG. 4 is a cross-sectional view of a convergence section of conduits in the endoscope reprocessor according to the first embodiment.

FIG. 5 is a flowchart for describing an example of a flow of cleaning processes for the endoscope reprocessor of the first embodiment.

FIG. 6 is a flowchart for describing another example of the flow of the cleaning processes for the endoscope reprocessor of the first embodiment.

FIG. 7 is a flowchart showing an example of a timing of switching between feeding liquid at low pressure and feeding liquid at high pressure in the cleaning processes for the endoscope reprocessor.

FIG. 8 is a flowchart showing another example of the timing of switching between feeding liquid at low pressure and feeding liquid at high pressure in the cleaning processes for the endoscope reprocessor.

FIG. 9 is a flowchart for describing another example of the flow of the cleaning processes for the endoscope reprocessor of the first embodiment.

FIG. 10 is a flowchart for describing an example of a flow of cleaning processes for the endoscope reprocessor of a second embodiment.

FIG. 11 is a flowchart for describing another example of the flow of the cleaning processes for the endoscope reprocessor of the second embodiment.

### DESCRIPTION OF EMBODIMENTS

An endoscope reprocessor 1 of embodiments of the present disclosure will be described below using the drawings. Hereinafter, the endoscope reprocessor 1 will be referred to as a reprocessor 1.

It is noted that the drawings based on the embodiment are schematic. The relationships between the thickness and width of each part and the ratio of the thickness of each part differ from the actual ones. The drawings also contain parts in which dimensional relationships and ratios are different from each other.

### First Embodiment

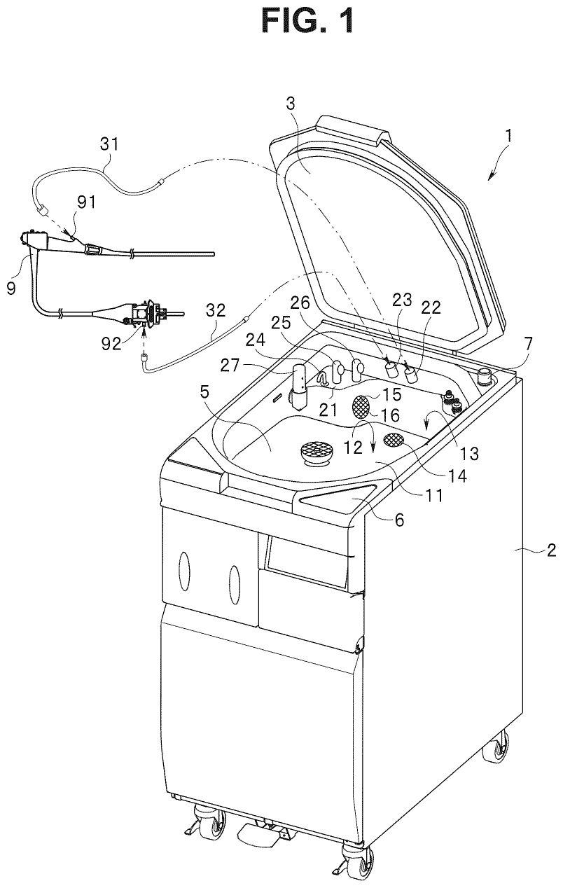

FIG. 1 is a perspective view of an endoscope reprocessor according to the first embodiment. As shown in FIG. 1, the reprocessor 1 includes a main body 2 and a top cover 3 which is openable and closable. FIG. 1 shows a state where the top cover 3 of the reprocessor 1 is open.

The reprocessor 1 is an apparatus for performing a restoration process on an endoscope 9 or an endoscope accessory. The restoration process may be rinsing with water, cleaning to eliminate contaminants such as organic matter, disinfection to inactivate predetermined microorganisms, sterilization to eliminate or kill all microorganisms, or any combination of these.

The main body 2 includes, at the top thereof, a processing tank 5 for performing a process such as cleaning and disinfection of the endoscope 9, an operation panel 6, and a water supply hose connection port 7.

The processing tank 5 stores a liquid such as a cleaning solution, water, an alcohol disinfectant solution, or a sterilization solution. The processing tank 5 includes an endoscope placement section 11 and a terrace 21.

The endoscope placement section 11 includes a bottom surface 12 and a side surface 13, is capable of placing the endoscope 9, and stores liquid. A discharge port 14 for discharging the stored liquid is provided in the bottom surface 12 of the endoscope placement section 11. A circulation port 16 having a mesh filter 15 is provided in the side surface 13 of the endoscope placement section 11. The circulation port 16 is in communication with a liquid pump 51 mentioned later. The circulation port 16 may be provided in the bottom surface 12.

The terrace 21 is provided at a position adjacent to the endoscope placement section 11 and higher than the endoscope placement section 11. The terrace 21 includes a forceps plug port 22 which is a first tube connection port, a suction ferrule port 23 which is a second tube connection port, a cleaning agent nozzle 24, a disinfectant solution nozzle 25, a water supply nozzle 26, and a water level sensor 27.

The forceps plug port 22 is a port to which a first tube 31 is to be connected. The suction ferrule port 23 is a port to which a second tube 32 is to be connected. The first tube 31 and the second tube 32 configure a cleaning tube. The number of ports that the reprocessor 1 has is not limited to 2.

The cleaning agent nozzle 24 supplies a cleaning solution into the processing tank 5. The disinfectant solution nozzle 25 supplies a disinfectant solution into the processing tank 5. The water supply nozzle 26 supplies water taken in from the water supply hose connection port 7 into the processing tank 5, and also supplies the liquid in the processing tank 5 taken in from the circulation port 16 with the mesh filter 15 back into the processing tank 5, thereby circulating the liquid. The mesh filter 15 filters contaminants P from the liquid. The water level sensor 27 detects a water level of the liquid stored in the processing tank 5.

The operation panel 6 is placed at a front section on top of the reprocessor 1. The operation panel 6 includes various operation buttons, not shown. A user gives various instructions to the reprocessor 1 via the operation panel 6.

The water supply hose connection port 7 is provided at a rear section on top of the reprocessor 1. A water supply hose connected to a water faucet, not shown, is connected to the water supply hose connection port 7, and water is supplied into the reprocessor 1 via the water supply nozzle 26.

The top cover 3 is provided openably and closably on the top of the main body 2. In the reprocessor 1, by bringing the top cover 3 into an opened state, the endoscope 9 can be placed on the endoscope placement section 11, and connection between the endoscope 9 and the reprocessor 1 can be established by means of the first tube 31 and the second tube 32. The reprocessor 1 is in a state where a process such as cleaning and disinfection is possible by bringing the top cover 3 into a closed state after the endoscope 9 is set.

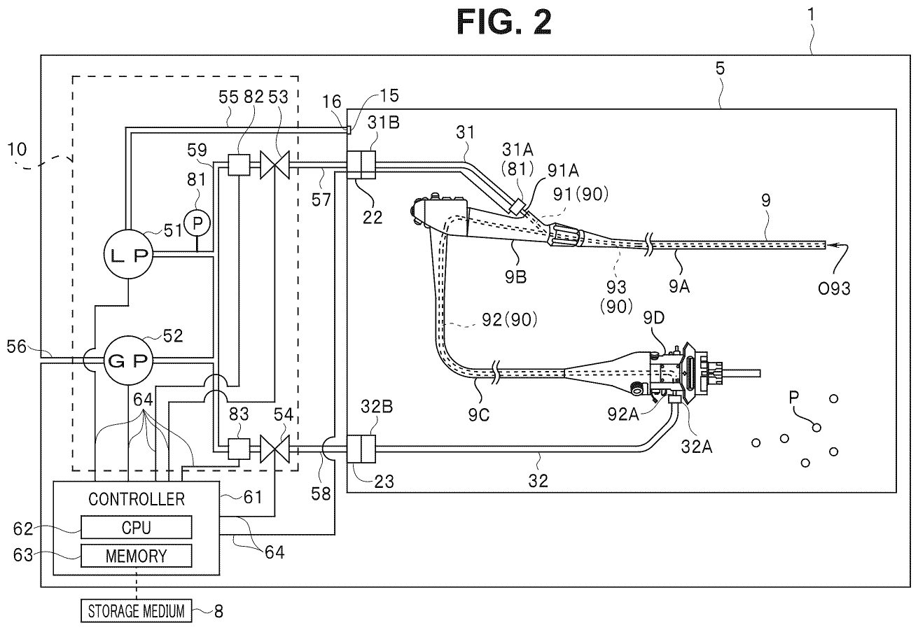

FIG. 2 is a configuration diagram of a main part of the endoscope reprocessor according to the first embodiment. FIG. 2 shows a state where the endoscope 9 to which the first tube 31 and the second tube 32 are connected is housed in the reprocessor 1. It is noted that FIG. 2 shows only the main configuration of the present disclosure. The reprocessor 1 may have a configuration different from that shown in FIG. 2 as long as it has the same functions as the configuration shown in FIG. 2.

The endoscope 9 includes an insertion portion 9A to be inserted into a body, an operation portion 9B, a universal cord 9C, and an endoscope connector 9D. The endoscope 9 includes a conduit 90 inside thereof. The conduit 90 includes a first conduit 91, a second conduit 92, and a third conduit 93. One end of the first conduit 91 is provided with a forceps ferrule 91A, and the other end converges with the second conduit 92. One end of the second conduit 92 is provided with a suction ferrule 92A, and the other end converges with the first conduit 91.

The third conduit 93, where the first conduit 91 and the second conduit 92 converge, passes through the insertion portion 9A and has an opening O93 at a distal end. A treatment instrument such as a forceps inserted from the forceps ferrule 91A passes through the first conduit 91 and the third conduit 93, and a distal end of the treatment instrument protrudes from the opening O93. A fluid fed from the suction ferrule 92A is emitted from the opening O93 via the second conduit 92 and the third conduit 93.

The first tube 31 includes a first connector 31A provided at one end thereof and connected to the forceps ferrule 91A which is a first ferrule of the endoscope 9, and a connector 31B provided at the other end thereof and connected to the forceps plug port 22 of the reprocessor 1. The forceps plug port 22 is in communication with the forceps ferrule 91A of the endoscope 9 via the first tube 31.

The second tube 32 includes a second connector 32A provided at one end thereof and connected to the suction ferrule 92A which is a second ferrule of the endoscope 9, and a connector 32B provided at the other end thereof and connected to the suction ferrule port 23 of the reprocessor 1. The suction ferrule port 23 is in communication with the suction ferrule 92A of the endoscope 9 via the second tube 32.

The reprocessor 1 includes the liquid pump 51, a gas pump 52, the processing tank 5, a first solenoid valve 53, a second solenoid valve 54, a pressure sensor 81, pressure control sections 82 and 83, the forceps plug port 22, the suction ferrule port 23, and a controller 61.

The liquid pump 51 takes in liquid such as cleaning solution in the processing tank 5 from the circulation port 16 having the mesh filter 15 into a conduit 55, pressurizes the taken-in liquid at a predetermined pressure, and feeds the pressurized liquid to a branch conduit 59. Although not shown, a part of the liquid taken in from the circulation port 16 is fed to the processing tank 5 by another pump. The mesh filter 15 filters the contaminants P that flow down from the conduit 90 of the endoscope 9 and float in the liquid in the processing tank 5.

The liquid pump 51 is connected to the first solenoid valve 53 and the second solenoid valve 54 via the branch conduit 59. The first solenoid valve 53 is connected to the forceps plug port 22 via a conduit 57. The second solenoid valve 54 is connected to the suction ferrule port 23 via a conduit 58.

The gas pump 52 takes in gas via a conduit 56, pressurizes the taken-in gas at a predetermined pressure, and feeds the pressurized gas to the branch conduit 59. The gas is air, for example. The gas pump 52 is connected to the first solenoid valve 53 and the second solenoid valve 54 via the branch conduit 59.

The pressure sensor 81 and the pressure control sections 82 and 83 are disposed in the branch conduit 59. The pressure control section 82 is disposed in the branch conduit 59 between the liquid pump 51 and the first solenoid valve 53. The pressure control section 83 is disposed in the branch conduit 59 between the liquid pump 51 and the second solenoid valve 54.

The pressure control sections 82 and 83 are configured of, for example, proportional valves or the like. The pressure control sections 82 and 83 control a pressure of the fluid to be supplied to the first conduit 91 and a pressure of the fluid to be supplied to the second conduit 92 according to a control signal from the controller 61.

The controller 61 includes a CPU 62, which is a central processing unit of a computer, and a memory 63 including a ROM, a RAM, or the like. Functions of the controller 61 are realized by the CPU 62 reading and executing a program from the memory 63. A program for causing the computer to execute a reprocessing process stored in the memory 63 is retained in a non-temporary computer-readable storage medium 8, and may be transferred to the memory 63.

The controller 61 further includes connection interfaces 64. The controller 61 is electrically connected to the liquid pump 51, the gas pump 52, the first solenoid valve 53, the second solenoid valve 54, the pressure sensor 81, and the pressure control sections 82 and 83 via respective connection interfaces 64. Each connection interface 64 may include an electrical line and a connector through which control signals are transmitted between the controller 61 and a respective one of the connected elements such as the liquid pump 51, the gas pump 52, the first solenoid valve 53, the second solenoid valve 54, the pressure sensor 81, and the pressure control sections 82 and 83.

To supply liquid to the conduit 90, the controller 61 activates the liquid pump 51 and stops the gas pump 52. To supply gas to the conduit 90, the controller 61 stops the liquid pump 51 and activates the gas pump 52.

To supply a gas-liquid two-phase flow to the conduit 90, the controller 61 activates the liquid pump 51 and then activates the gas pump 52. The gas-liquid two-phase flow refers to a state in which liquid and gas are mixed in the conduit 90 of the endoscope 9 by feeding air to the conduit 90 from the gas pump 52 in a state where the conduit 90 is filled with liquid. More specifically, the gas-liquid two-phase flow includes any of a state in which gas bubbles are present in the liquid, a state in which liquid droplets are present in the gas, and a state in which liquid slugs and gas pockets are present adjacent to one another.

The controller 61 controls an opening or closing state of the first solenoid valve 53 so that a fluid at a predetermined flow rate (predetermined pressure) is supplied to the first conduit 91 via the first tube 31. The controller 61 controls an opening or closing state of the second solenoid valve 54 so that a fluid at a predetermined flow rate is supplied to the second conduit 92 via the second tube 32.

The controller 61 controls the pressure control sections 82 and 83, thereby controlling the pressures of the fluids to be supplied to the first conduit 91 and the second conduit 92. The controller 61 controls the pressure control sections 82 and 83 to supply fluid to the first conduit 91 and the second conduit 92 at a first pressure, or to supply fluid to the first conduit 91 and the second conduit 92 at a second pressure higher than the first pressure.

The liquid pump 51, the gas pump 52, the first solenoid valve 53, and the second solenoid valve 54, and the pressure control sections 82 and 83 configure a fluid supply unit (hereinafter may be referred to as a “fluid supply assembly”) 10. The controller 61 controls timing and pressure at which the fluid supply unit 10 supplies fluid to the conduit 90.

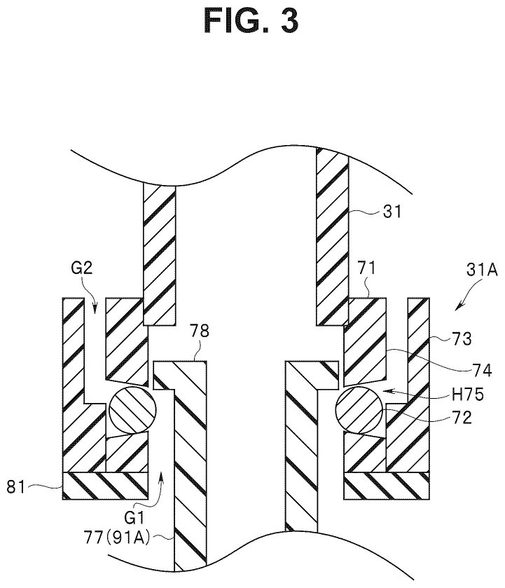

FIG. 3 is a cross-sectional view of a joint section between the first connector and the forceps ferrule in the endoscope reprocessor according to the first embodiment.

The first connector 31A includes a connector main body 71 provided at a distal end of the first tube 31, a plurality of spheres 72, and a connector cover 73 provided on an outer circumferential section of the connector main body 71.

The connector main body 71 is made of plastic or the like. The connector main body 71 is tubular, and includes a plurality of circular holes H75 in a circumferential side portion 74. For example, four holes H75 are provided in the circumferential side portion 74 of the connector main body 71 at equal intervals along a circumferential direction. Each of the holes H75 has a decreasing diameter from an outer surface toward an inner surface of the connector main body 71, and has a tapered cross section in a thickness direction of the circumferential side portion 74.

The plurality of spheres 72 is configured of metal or the like. The plurality of spheres 72, each having a diameter larger than that of the hole H75 in an inner circumferential surface of the circumferential side portion 74, are placed with a part fitted in the holes H75, so as not to fall off from the inner circumferential surface of the circumferential side portion 74.

The connector cover 73 is configured of plastic or the like. The connector cover 73 is placed on an outside of the connector main body 71 where the plurality of spheres 72 is placed. A circumferential gap G2 is formed between the connector cover 73 and the connector main body 71.

The forceps ferrule 91A is made of metal, resin, or the like. The forceps ferrule 91A has a body section 77 formed in a cylindrical shape, and has an outwardly extending flange 78 at a distal end.

The first connector 31A of the first tube 31 is detachably attached to the forceps ferrule 91A. In a state where the first connector 31A is attached to the forceps ferrule 91A, the plurality (four, in this case) of spheres 72 of the first connector 31A engage with the outwardly extending flange 78 of the forceps ferrule 91A to inhibit detachment of the flange 78. A circumferential gap G1 is formed between the body section 77 of the forceps ferrule 91A and the connector main body 71.

The fluid fed from the first tube 31 is introduced into an inner side of the body section 77 of the forceps ferrule 91A, flows out from the gap G2 via a space between the connector main body 71 in which the hole H75 is formed and the sphere 72, and flows out from the gap G1 via a space between the connector main body 71 and the forceps ferrule 91A. The fluid flowing out from the gaps G1 and G2 in a connection area between the first connector 31A and the forceps ferrule 91A cleans the forceps ferrule 91A.

It is noted that the shape of the first connector 31A of the first tube 31 is not limited to that mentioned above, and a connector disclosed in the international publication WO2015-001843 already described may also be employed.

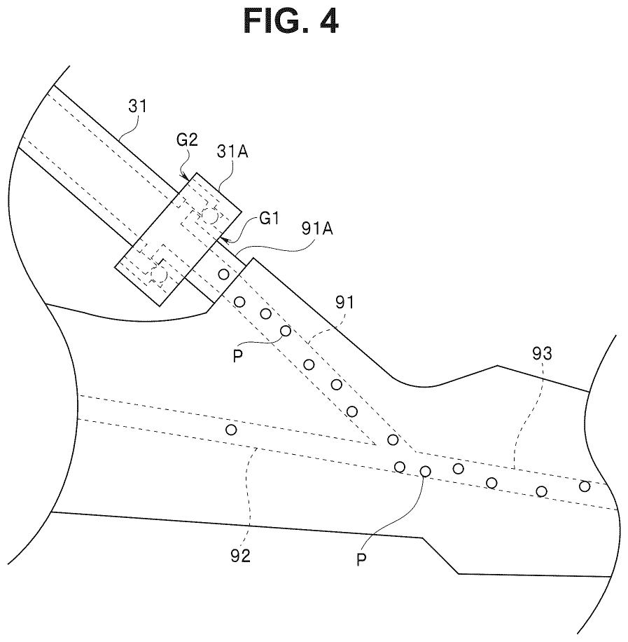

FIG. 4 is a cross-sectional view of a convergence section of the conduits in the endoscope reprocessor according to the first embodiment. As shown in FIG. 4, the contaminants P may adhere to the conduit 90 of the endoscope 9 after use, particularly to the first conduit 91 and the third conduit 93, which are withdrawal paths for the treatment instrument inserted into the body.

As already described, since the gaps G1 and G2 are present between the first connector 31A of the first tube 31 connected to the forceps ferrule 91A and the forceps ferrule 91A, if, in the reprocessing process, the fluid supplied to the second conduit 92 flows back through the first conduit 91 via the convergence section, the contaminants P flushed away by the fluid may be trapped in the gaps G1 and G2. In particular, if gas and liquid are fed at high pressure to the first conduit 91 and the second conduit 92 from the start, large contaminants P may be trapped in the gaps G1 and G2 between the first connector 31A and the forceps ferrule 91A.

In the present embodiment, liquid is fed first at low pressure to the first conduit 91 and the second conduit 92 to remove the large contaminants P adhering inside the conduit 90, and then fed at high pressure. This inhibits the large contaminants P from being trapped in the gaps G1 and G2 between the first connector 31A and the forceps ferrule 91A.

Next, cleaning processes as operations of the endoscope reprocessor 1 will be described. The user opens the top cover 3 of the endoscope reprocessor 1, and sets an endoscope 9 to be cleaned in the endoscope reprocessor 1. Specifically, the user connects the connector 31B of the first tube 31 to the forceps plug port 22, connects the first connector 31A to the forceps ferrule 91A of the endoscope 9, connects the connector 32B of the second tube 32 to the suction ferrule port 23, and connects the second connector 32A to the suction ferrule 92A of the endoscope 9. It is noted that, although not shown in the figures, in addition to the connection by the first tube 31 and the second tube 32, connection between the endoscope reprocessor 1 and the endoscope 9 is established by tubes, as necessary.

After connecting the endoscope reprocessor 1 and the endoscope 9, the user places the endoscope 9 in the endoscope placement section 11 and brings the top cover 3 into the closed state.

When the user gives a start instruction for a process such as cleaning and disinfection from the operation panel 6, the CPU 62 reads a predetermined program from the memory 63 and starts the process of the program.

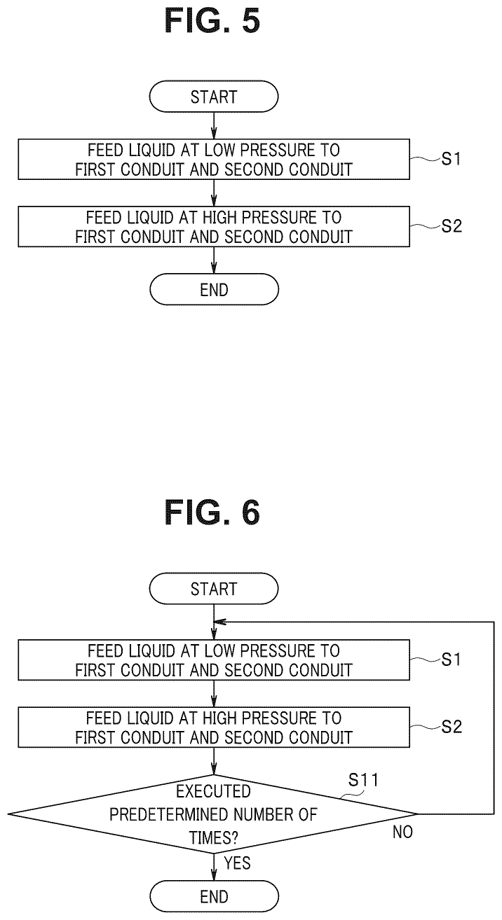

FIG. 5 is a flowchart for describing an example of a flow of the cleaning processes for the endoscope reprocessor of the first embodiment.

The controller 61 controls the fluid supply unit 10 to feed liquid at low pressure to the first conduit 91 and the second conduit 92 (S1). This process removes the large contaminants P adhering inside the conduit 90, especially in the vicinity of the forceps ferrule 91A.

Next, the controller 61 controls the fluid supply unit 10 to feed liquid at high pressure to the first conduit 91 and the second conduit 92 (S2), and ends the cleaning processes.

As described above, in the cleaning processes of the present embodiment, liquid is fed first at low pressure and then at high pressure to the first conduit 91 and the second conduit 92. As mentioned above, if liquid is fed at high pressure from the start, the large contaminants P flushed away by backflow may be trapped in the gaps G1 and G2. In contrast, in the cleaning processes of the present embodiment, liquid is first fed at low pressure to remove the large contaminants P adhering inside the conduit 90. After the large contaminants P are removed, liquid is fed at high pressure to clean inside the conduit 90, thereby inhibiting the large contaminants P from being trapped in the gaps G1 and G2.

Therefore, according to the endoscope reprocessor 1 of the present embodiment, it is possible to suppress clogging of the space between a ferrule of a forceps port and the connector of the cleaning tube with contaminants.

It is noted that the cleaning processes of the present embodiment are not limited to those shown in FIG. 5.

FIG. 6 is a flowchart for describing another example of the flow of the cleaning processes for the endoscope reprocessor of the first embodiment. It is noted that, in FIG. 6, the same reference numerals are attached to the same processes as in FIG. 5, and descriptions thereof will be omitted.

After feeding liquid at high pressure to the first conduit 91 and the second conduit 92 in the process in S2, the controller 61 determines whether the cleaning processes in S1 and S2 have been executed a predetermined number of times (S11). If determining that the cleaning processes have not been executed the predetermined number of times (S11: NO), the controller 61 returns control to the cleaning process in S1 and repeats the same processes. On the other hand, if determining that the cleaning processes have been executed the predetermined number of times (S11: YES), the controller 61 ends the cleaning processes.

By finely switching between feeding liquid at low pressure and feeding liquid at high pressure, water hammer caused by sudden pressure changes is generated to remove the contaminants P inside the conduit 90.

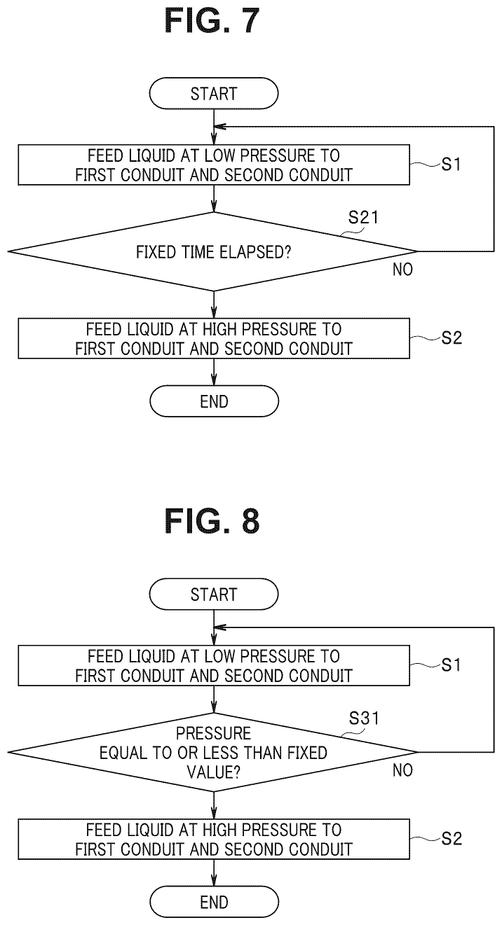

A timing of switching between feeding liquid at low pressure and feeding liquid at high pressure may be as shown in FIG. 7.

FIG. 7 is a flowchart showing an example of the timing of switching between feeding liquid at low pressure and feeding liquid at high pressure in the cleaning processes for the endoscope reprocessor. It is noted that, in FIG. 7, the same reference numerals are attached to the same processes as in FIG. 5, and descriptions thereof will be omitted.

Upon feeding liquid at low pressure to the first conduit 91 and the second conduit 92 in the process in S1, the controller 61 determines whether a fixed time has elapsed (S21).

If determining that the fixed time has not elapsed (S21: NO), the controller 61 returns control to the cleaning process in S1 and repeats the same process. On the other hand, if determining that the fixed time has elapsed (S21: YES), the controller 61 proceeds to the process in S2 to feed liquid at high pressure to the first conduit 91 and the second conduit 92.

It is noted that the timing of switching between feeding liquid at low pressure and feeding liquid at high pressure is not limited to that of the processes shown in FIG. 7.

FIG. 8 is a flowchart showing another example of the timing of switching between feeding liquid at low pressure and feeding liquid at high pressure in the cleaning processes for the endoscope reprocessor. It is noted that, in FIG. 8, the same reference numerals are attached to the same processes as in FIG. 5, and descriptions thereof will be omitted.

Upon feeding liquid at low pressure to the first conduit 91 and the second conduit 92 in the process in S1, the controller 61 determines whether the pressure has become equal to or less than a fixed value (S31). The controller 61 determines whether the pressure has become equal to or less than the fixed value based on a measurement result of the pressure sensor 81.

If determining that the pressure is not equal to or less than the fixed value (S31: NO), the controller 61 returns control to the cleaning process in S1 and repeats the same process. On the other hand, if determining that the pressure is equal to or less than the fixed value (S31: YES), the controller 61 proceeds to the process in S2 to feed liquid at high pressure to the first conduit 91 and the second conduit 92.

For example, when the large contaminants P adhere to the vicinity of the forceps ferrule 91A or inside the conduit 90, the pressure of the fed liquid increases. Then, when the large contaminants P are removed by feeding liquid at low pressure, the pressure decreases. Therefore, when the pressure has become equal to or less than the fixed value, the controller 61 determines that the large contaminants P have been removed, and switches from feeding liquid at low pressure to feeding liquid at high pressure.

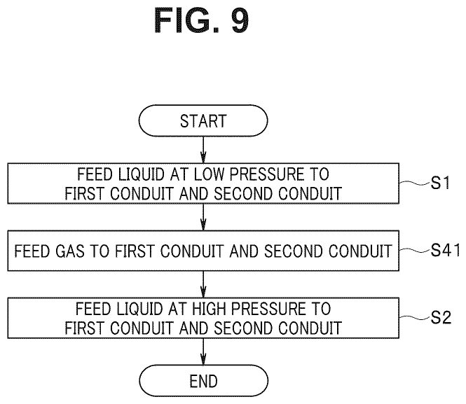

The cleaning processes of the present embodiment may also be those shown in FIG. 9.

FIG. 9 is a flowchart for describing another example of the flow of the cleaning processes for the endoscope reprocessor of the first embodiment. It is noted that, in FIG. 9, the same reference numerals are attached to the same processes as in FIG. 5, and descriptions thereof will be omitted.

After feeding liquid at low pressure to the first conduit 91 and the second conduit 92 in the process in S1, the controller 61 feeds gas to the first conduit 91 and the second conduit 92 (S41). Thereafter, in the process in S2, the controller 61 feeds liquid at high pressure to the first conduit 91 and the second conduit 92.

As in the above processes, after feeding liquid at low pressure, the feeding of liquid is not immediately switched to that in high pressure, but gas is fed once to the first conduit 91 and the second conduit 92. In this manner, after liquid inside the conduit 90 is blown off, liquid is fed at high pressure.

By feeding liquid in a state where the liquid inside the conduit 90 is blown off, the pressure on a distal end side of the conduit 90 (distal end side of the insertion portion 9A) decreases, and the flow velocity of the liquid when fed at high pressure increases. As a result, the ability to clean the inside of the conduit 90 can be improved.

### Second Embodiment

Next, the second embodiment will be described.

The configuration of the reprocessor 1 is the same as that in the first embodiment, but the cleaning processes are different from those in the first embodiment. The reprocessor 1 of the second embodiment performs cleaning processes using a gas-liquid two-phase flow.

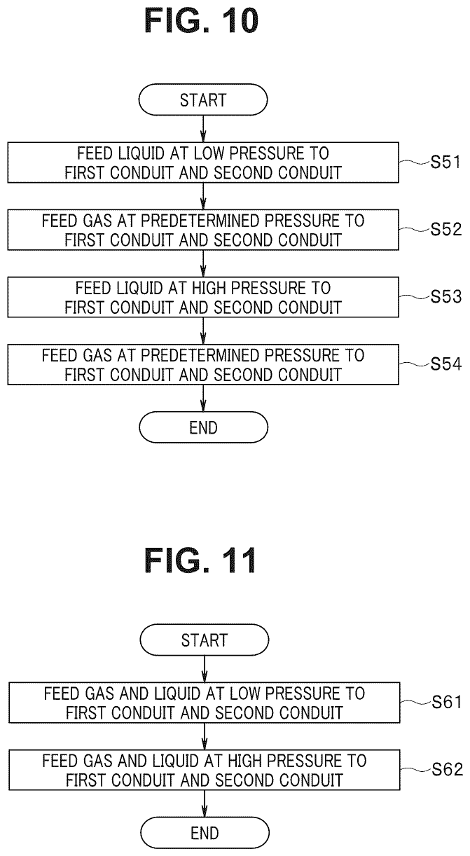

FIG. 10 is a flowchart for describing an example of a flow of cleaning processes for the endoscope reprocessor of the second embodiment.

The controller 61 controls the fluid supply unit 10 to feed liquid at low pressure to the first conduit 91 and the second conduit 92 (S51). Next, the controller 61 controls the fluid supply unit 10 to feed gas at a predetermined pressure to the first conduit 91 and the second conduit 92 (S52).

The controller 61 then controls the fluid supply unit 10 to feed liquid at high pressure to the first conduit 91 and the second conduit 92 (S53). Finally, the controller 61 controls the fluid supply unit 10 to feed gas at a predetermined pressure to the first conduit 91 and the second conduit 92 (S54), and ends the cleaning processes. It is noted that the predetermined pressure in the process in S52 and the predetermined pressure in the process in S54 may be the same or different.

In the gas-liquid two-phase flow, liquid is fed from the liquid pump 51 into the conduit 90 to fill the conduit 90 with the liquid, and then in this state, air is fed from the gas pump 52, thereby mixing liquid and gas inside the conduit 90. At this time, liquid is fed at low pressure (first pressure) and gas is fed at a predetermined pressure (second pressure) to remove the large contaminants P, as in the first embodiment, and then liquid is fed at high pressure (third pressure) and gas is fed at a predetermined pressure (fourth pressure) to perform cleaning processes inside the conduit 90.

Thus, according to the endoscope reprocessor 1 of the present embodiment, as in the first embodiment, the clogging of the space between the ferrule of the forceps port and the connector of the cleaning tube with contaminants can be suppressed.

It is noted that the cleaning processes using the gas-liquid two-phase flow are not limited to those shown in FIG. 10.

FIG. 11 is a flowchart for describing another example of the flow of the cleaning processes for the endoscope reprocessor of the second embodiment.

The controller 61 controls the fluid supply unit 10 to feed gas and liquid at low pressure to the first conduit 91 and the second conduit 92 (S61). The controller 61 causes the liquid pump 51 and the gas pump 52 of the fluid supply unit 10 to operate simultaneously and controls the pressure control sections 82 and 83, thereby feeding gas and liquid at low pressure.

Next, the controller 61 controls the fluid supply unit 10 to feed gas and liquid at high pressure to the first conduit 91 and the second conduit 92 (S62). The controller 61 causes the liquid pump 51 and the gas pump 52 to operate simultaneously, and in this state, controls the pressure control sections 82 and 83, thereby feeding gas and liquid at high pressure.

In this way, the liquid pump 51 and the gas pump 52 may be operated simultaneously to supply the gas-liquid two-phase flow, in which liquid and gas are mixed, to the first conduit 91 and the second conduit 92. At this time, as in the first embodiment, gas and liquid are fed at low pressure to remove the large contaminants P, and then gas and liquid are fed at high pressure to perform cleaning processes of the conduit 90.

As a result, the large contaminants P can be inhibited from being trapped in the gaps G1 and G2 between the first connector 31A and the forceps ferrule 91A.

It is noted that the steps in the flowchart in the present specification may be executed in a changed order, executed simultaneously, or executed in a different order for each execution, unless contrary to the nature thereof.

The present disclosure is not limited to the above-mentioned embodiments, and it is needless to say that various modifications, combinations, and applications are possible without departing from the gist of the disclosure.

### General Interpretation Notes

The following applies throughout this specification and drawings.

It is noted that various connections are described between elements in the foregoing description. These connections, unless specified otherwise, may be either direct or indirect, and this specification is not intended to be limiting in that respect. Aspects of the present disclosure may be implemented using circuits (such as application-specific integrated circuits) or computer software stored on non-transitory computer-readable storage media, including but not limited to RAMs, ROMs, flash memories, EEPROMs, CD media, DVD media, temporary storage, hard disk drives, floppy drives, permanent storage, and the like.

As used herein, the term “processor” encompasses a single processor or a group of multiple processors, which may include a single-core processor, a multi-core processor, multiple processors within a single device, or multiple processors in wired or wireless communication with each other. Such processors may be locally or remotely distributed and may operate collaboratively or in a distributed fashion across a network of devices, the Internet, or the cloud to collectively perform the tasks attributed to the “processor” described herein. It should be understood that not all of the processors included in the system or device are necessarily involved in performing each operation attributed to the “processor.” Rather, only a subset of at least one processor may contribute to performing a particular operation. Furthermore, different subsets of at least one processor may contribute to performing different operations, and the composition of the subsets may vary from one operation to another. Similarly, the term “non-transitory computer-readable (storage) medium” encompasses a single storage medium or a group of multiple storage media, which may be locally or remotely distributed and may collectively store and provide access to instructions, data, or other information in a coordinated or distributed manner.

In the present disclosure, an inclusive OR—meaning that it includes either A, B, or both—may be expressed as “A and/or B,” “at least one of A or B,” or “at least one selected from the group consisting of A and B.” Additionally, the expressions “one of A or B” and “either A or B,” as used herein, refer to a case where A or B is selected exclusively, but not both. The same interpretation applies in cases where three or more selectable elements are considered.

Non-limiting examples according to aspects of the present disclosure will be described in the following clauses:

Clause 1: An endoscope reprocessor comprising:
a first tube including a first connector connected to a first ferrule of an endoscope;
a second tube including a second connector connected to a second ferrule of the endoscope;
a fluid supply unit that supplies fluid to a conduit of the endoscope via each of the first tube and the second tube; and
a controller that controls the fluid supply unit, wherein
the conduit includes a first conduit in communication with the first ferrule, a second conduit in communication with the second ferrule, and a third conduit where the first conduit and the second conduit converge, the third conduit including an opening at a distal end of an insertion portion of the endoscope, and
the controller controls the fluid supply unit to feed liquid at a first pressure to the first conduit and the second conduit, and then feed the liquid at a second pressure higher than the first pressure.

Clause 2: The endoscope reprocessor according to clause 1, wherein the controller controls the fluid supply unit to execute a process of feeding the liquid at the first pressure and then feeding the liquid at the second pressure, a predetermined number of times.

Clause 3: The endoscope reprocessor according to clause 1, wherein the controller controls the fluid supply unit to feed the liquid at the first pressure, and upon determining that a fixed time elapses, feed the liquid at the second pressure.

Clause 4: The endoscope reprocessor according to clause 1, wherein the controller controls the fluid supply unit to feed the liquid at the first pressure, and upon determining that the pressure becomes equal to or less than a fixed value, feed the liquid at the second pressure.

Clause 5: The endoscope reprocessor according to clause 1, wherein the controller controls the fluid supply unit to feed the liquid at the first pressure, then feed gas to the first conduit and the second conduit, and after the feeding of the gas, feed the liquid at the second pressure.

Clause 6: The endoscope reprocessor according to clause 1, wherein the controller controls the fluid supply unit to feed liquid at the first pressure to the first conduit and the second conduit, feed gas at a third pressure, feed the liquid at the second pressure higher than the first pressure, and feed the gas at a fourth pressure.

Clause 7: The endoscope reprocessor according to clause 1, wherein the controller controls the fluid supply unit to feed gas and liquid at the first pressure to the first conduit and the second conduit, and then feed the gas and the liquid at the second pressure higher than the first pressure.

Clause 8: A method of operating an endoscope reprocessor, the method comprising performing a control with respect to an endoscope, wherein
the endoscope comprises:
   a first conduit in communication with a first ferrule;
   a second conduit in communication with a second ferrule; and
   a third conduit where the first conduit and the second conduit converge,
   wherein the third conduit includes an opening at a distal end of an insertion portion of the endoscope, and
   controlling to feed liquid at a first pressure to the first conduit and the second conduit, and then feed the liquid at a second pressure higher than the first pressure.

Clause 9: The method of operating the endoscope reprocessor according to clause 8, the method further comprising:
controlling to execute a process of feeding the liquid at the first pressure and then feeding the liquid at the second pressure, a predetermined number of times.

Clause 10: The method of operating the endoscope reprocessor according to clause 8, the method further comprising:
controlling to feed the liquid at the first pressure, and upon determining that a fixed time elapses, feed the liquid at the second pressure.

Clause 11: The method of operating the endoscope reprocessor according to clause 8, the method further comprising:
controlling to feed the liquid at the first pressure, and upon determining that the pressure becomes equal to or less than a fixed value, feed the liquid at the second pressure.

Clause 12: The method of operating the endoscope reprocessor according to clause 8, the method further comprising:
controlling to feed the liquid at the first pressure, then feed gas to the first conduit and the second conduit, and after the feeding of the gas, feed the liquid at the second pressure.

Clause 13: The method of operating the endoscope reprocessor according to clause 8, the method further comprising:
controlling to feed liquid at the first pressure to the first conduit and the second conduit, feed gas at a third pressure, feed the liquid at the second pressure higher than the first pressure, and feed the gas at a fourth pressure.

Clause 14: The method of operating the endoscope reprocessor according to clause 8, the method further comprising:
controlling to feed gas and liquid at the first pressure to the first conduit and the second conduit, and then feed the gas and the liquid at the second pressure higher than the first pressure.

Clause 15: A control apparatus comprising a processor controlling with respect to an endoscope, wherein
the endoscope comprises:
   a first conduit in communication with a first ferrule;
   a second conduit in communication with a second ferrule; and
   a third conduit where the first conduit and the second conduit converge,
wherein the third conduit includes an opening at a distal end of an insertion portion of the endoscope, and
controlling to feed liquid at a first pressure to the first conduit and the second conduit, and then feed the liquid at a second pressure higher than the first pressure.

Clause 16: A control apparatus according to clause 15, wherein
the control apparatus controls to execute a process of feeding the liquid at the first pressure and then feeding the liquid at the second pressure, a predetermined number of times.

Clause 17: A control apparatus according to clause 15, wherein
the control apparatus controls to feed the liquid at the first pressure, and upon determining that a fixed time elapses, feed the liquid at the second pressure.

Clause 18: A control apparatus according to clause 15, wherein
the control apparatus controls to feed the liquid at the first pressure, and upon determining that the pressure becomes equal to or less than a fixed value, feed the liquid at the second pressure.

Clause 19: A control apparatus according to clause 15, wherein
the control apparatus controls to feed the liquid at the first pressure, then feed gas to the first conduit and the second conduit, and after the feeding of the gas, feed the liquid at the second pressure.

Clause 20: A control apparatus according to clause 15, wherein
the control apparatus controls to feed liquid at the first pressure to the first conduit and the second conduit, feed gas at a third pressure, feed the liquid at the second pressure higher than the first pressure, and feed the gas at a fourth pressure.

What is claimed is:

1. An endoscope reprocessor comprising:
a first tube connectable to a first ferrule of an endoscope;
a second tube connectable to a second ferrule of the endoscope;
a fluid supply assembly configured to supply fluid to a conduit of the endoscope via the first tube and the second tube, wherein the conduit includes a first conduit communicating with the first ferrule, and a second conduit communicating with the second ferrule, the first and second conduits converging into a third conduit having an opening at a distal end of an insertion portion of the endoscope; and
a controller configured to control the fluid supply assembly to feed liquid to the first and second conduits at a first pressure, and then feed liquid to the first and second conduits at a second pressure higher than the first pressure.

2. The endoscope reprocessor according to claim 1,
wherein the controller is further configured to control the fluid supply assembly to execute, a predetermined number of times, a process of feeding liquid at the first pressure and then feeding liquid at the second pressure.

3. The endoscope reprocessor according to claim 1,
wherein the controller is further configured to control the fluid supply assembly to feed liquid to the first conduit and the second conduit at the second pressure in response to determining that a particular time has elapsed after the feeding of liquid to the first conduit and the second conduit at the first pressure.

4. The endoscope reprocessor according to claim 1,
wherein the controller is further configured to control the fluid supply assembly to feed liquid to the first conduit and the second conduit at the second pressure in response to determining that the pressure has become equal to or less than a particular value after the feeding of liquid to the first conduit and the second conduit at the first pressure.

5. The endoscope reprocessor according to claim 1,
wherein the controller is further configured to control the fluid supply assembly to:
feed liquid to the first conduit and the second conduit at the first pressure;
then feed gas to the first conduit and the second conduit; and
thereafter feed liquid to the first conduit and the second conduit at the second pressure.

6. The endoscope reprocessor according to claim 1,
wherein the controller is further configured to control the fluid supply assembly to:
feed liquid to the first conduit and the second conduit at the first pressure;
then feed gas to the first conduit and the second conduit at a third pressure;
then feed liquid to the first conduit and the second conduit at the second pressure higher than the first pressure; and
thereafter feed gas to the first conduit and the second conduit at a fourth pressure.

7. The endoscope reprocessor according to claim 1,
wherein the controller is further configured to control the fluid supply assembly to feed gas and liquid to the first conduit and the second conduit at the first pressure, and then feed gas and liquid to the first conduit and the second conduit at the second pressure higher than the first pressure.

8. A control method implementable by an endoscope reprocessor configured to supply fluid to a conduit of an endoscope via a first tube and a second tube, the method comprising:
feeding liquid to a first conduit and a second conduit at a first pressure; and
then feeding liquid to the first conduit and the second conduit at a second pressure higher than the first pressure,
wherein the endoscope comprises:
a first ferrule connectable to the first tube;
a second ferrule connectable to the second tube; and
the conduit including the first conduit communicating with the first ferrule, and the second conduit communicating with the second ferrule, the first and second conduits converging into a third conduit having an opening at a distal end of an insertion portion of the endoscope.

9. The control method according to claim 8,
wherein a process of feeding liquid to the first conduit and the second conduit at the first pressure and then feeding liquid to the first conduit and the second conduit at the second pressure is executed a predetermined number of times.

10. The control method according to claim 8,
wherein the feeding of liquid to the first conduit and the second conduit at the second pressure is executed in response to a determination that a particular time has elapsed after the feeding of liquid to the first conduit and the second conduit at the first pressure.

11. The control method according to claim 8,
wherein the feeding of liquid to the first conduit and the second conduit at the second pressure is executed in response to a determination that the pressure has become equal to or less than a particular value after the feeding of liquid to the first conduit and the second conduit at the first pressure.

12. The control method according to claim 8, further comprising:
feeding gas to the first conduit and the second conduit after feeding liquid to the first conduit and the second conduit at the first pressure and before feeding liquid to the first conduit and the second conduit at the second pressure.

13. The control method according to claim 8, further comprising:
feeding gas to the first conduit and the second conduit at a third pressure after feeding liquid to the first conduit and the second conduit at the first pressure; and
feeding gas to the first conduit and the second conduit at a fourth pressure after feeding liquid to the first conduit and the second conduit at the second pressure higher than the first pressure.

14. The control method according to claim 8,
wherein a process of feeding liquid to the first conduit and the second conduit at the first pressure and then feeding liquid to the first conduit and the second conduit at the second pressure higher than the first pressure includes:
feeding gas and liquid to the first conduit and the second conduit at the first pressure and then feeding gas and liquid to the first conduit and the second conduit at the second pressure higher than the first pressure.

15. A control apparatus comprising:
a connection interface connectable to a fluid supply assembly configured to supply fluid to a conduit of an endoscope via a first tube and a second tube; and
a processor configured to control the fluid supply assembly to feed liquid to a first conduit and a second conduit at a first pressure, and then feed liquid to the first and second conduits at a second pressure higher than the first pressure,
wherein the endoscope comprises:
a first ferrule connectable to the first tube;
a second ferrule connectable to the second tube; and
the conduit including the first conduit communicating with the first ferrule, and the second conduit communicating with the second ferrule, the first and second conduits converging into a third conduit having an opening at a distal end of an insertion portion of the endoscope.

16. The control apparatus according to claim 15,
wherein the processor is further configured to control the fluid supply assembly to execute, a predetermined number of times, a process of feeding liquid to the first conduit and the second conduit at the first pressure and then feeding liquid to the first conduit and the second conduit at the second pressure.

17. The control apparatus according to claim 15,
wherein the processor is further configured to control the fluid supply assembly to feed liquid to the first conduit and the second conduit at the second pressure in response to determining that a particular time has elapsed after the feeding of liquid to the first conduit and the second conduit at the first pressure.

18. The control apparatus according to claim 15,
wherein the processor is further configured to control the fluid supply assembly to feed liquid to the first conduit and the second conduit at the second pressure in response to determining that the pressure has become equal to or less than a particular value after the feeding of liquid to the first conduit and the second conduit at the first pressure.

19. The control apparatus according to claim 15,
wherein the processor is further configured to control the fluid supply assembly to:
feed liquid to the first conduit and the second conduit at the first pressure;
then feed gas to the first conduit and the second conduit; and
thereafter feed liquid to the first conduit and the second conduit at the second pressure.

20. The control apparatus according to claim 15,
wherein the processor is further configured to control the fluid supply assembly to:
feed liquid to the first conduit and the second conduit at the first pressure;
then feed gas to the first conduit and the second conduit at a third pressure;
then feed liquid to the first conduit and the second conduit at the second pressure higher than the first pressure; and
thereafter feed gas to the first conduit and the second conduit at a fourth pressure.
